(19) United States
(12) Patent Application Publication
(10) Pub. No.: US 2005/0090817 A1
(43) Pub. Date: Apr. 28, 2005

(54) Bendable endoscopic bipolar device

(75) Inventor: Huy D. Phan, San Jose, CA (US)

Correspondence Address: Bingham McCutchen, LLP, Suite1800, Three Embarcadero, San Francisco, CA, 94111-4067, US

(73) Assignee: Scimed Life Systems, Inc.

(21) Appl. No.: 10/692,241

(22) Filed: Oct. 22, 2003

(51) Int. Cl. A61B 18/14

(52) U.S. Cl. 606/41; 606/51

(57) ABSTRACT

An ablation device for creating a lesion within a body is provided. The ablation device includes a shaft having a proximal end and a distal end, wherein at least a portion of the shaft is bendable to form a desired configuration. A clamp assembly is secured to the distal end of the shaft. The clamp assembly has a first jaw and a second jaw, the second jaw moveable relative to the first jaw to open or close the clamp assembly. The ablation device also includes a first electrode secured to the first jaw of the clamp assembly, a second electrode secured to the second jaw of the clamp assembly, and a handle connected to the proximal end of the shaft.

### FIELD OF THE INVENTION

The present invention pertains to devices and methods for ablating tissue, and more particularly, to ablation devices and methods for creating transmural lesions within a body.

### BACKGROUND

Physicians use catheters today in medical procedures to gain access into interior regions of the body, e.g., to ablate targeted tissue areas. For example, in electrophysiological therapy, ablation is used to treat cardiac rhythm disturbances. During these procedures, a physician steers a catheter through a main vein or artery into the interior region of the heart that is to be treated. The physician places an ablating element carried on the catheter near the targeted cardiac tissue, and directs energy from the ablating element to ablate the tissue and form a lesion. Such procedures may be used to treat arrhythmia, a condition in the heart in which abnormal electrical signals are generated in the heart tissue.

In certain procedures, instead of accessing tissue through a vein or an artery, it may be desirable to access the tissue directly through a patient's skin. For example, atrial tissue may be ablated by making an incision in a patient's skin, and inserting an ablation device through the incision to access the atrial tissue. This may require using an ablation clamp that includes a shaft and a clamping device carried at a distal end of the shaft for holding tissue. One problem associated with such ablation clamps is that the shaft may have a predetermined profile, and may be substantially rigid. As a result, a physician may need to determine the location of the incision point with sufficient accuracy such that, when the ablation device is inserted into the patient through the incision, the clamping device may reach the target tissue site. In addition, when a different target tissue site needs to be ablated, the physician may need to use a different ablation clamp having a shaft with different shape and/or may need to access the site via another incision. Thus, the procedure may become complicated, requiring exchanging multiple surgical devices, which may be undesirable. In addition, an ablation clamp having a shaft shape that meets the encountered anatomy may not be readily available during a procedure.

Another problem associated with existing ablation clamps is that they may cause uneven compression of a target tissue. FIGS. 1A and 1B show an example of an existing ablation clamp 10 having a scissor-type configuration. Particularly, the ablation clamp 10 may include a first jaw 12 and a second jaw 14 rotatably connected to one another by a pin 16. The first and the second jaws 12, 14 carry first and second electrodes 17, 18, respectively, for treating a target tissue structure 20. When the ablation clamp 10 is closed to hold the tissue structure 20, a proximal portion 22 of the tissue structure 20 may be compressed more than a distal portion 24 (FIG. 1B), because a space d1 between the jaws 12, 14 of the ablation clamp 10 at the proximal portion 22 of the tissue structure 20 may be smaller than a space d2 between the jaws 12, 14 at the distal portion 24 of the tissue structure 20. As a result, a compression force on the tissue structure 20 may be greater at the proximal portion 22 than at the distal portion 24 when the tissue -structure 20 is held by the ablation clamp 10. Due to the greater force at the proximal portion 22, and the smaller space between the electrodes 17, 18 at the proximal portion 22 of the tissue structure 20, the proximal portion 22 of the tissue structure 20 may be subjected to higher intensity energy than the distal end 24, thereby creating an undesirable lesion.

Thus, improved apparatus and methods for creating lesions or otherwise treating tissue would be useful.

### SUMMARY OF THE INVENTION

In accordance with one aspect of the present invention, an apparatus for creating a lesion within a body is provided that may include a shaft having a proximal end and a distal end, at least a portion of the shaft being bendable to form a desired configuration. A clamp assembly may be carried by the distal end of the shaft that includes first and second opposing jaws, at least one of the jaws moveable relative to the other jaw to open and/or close the clamp assembly. The apparatus may also include an electrode on one or both of the jaws of the clamp assembly, and a handle on the proximal end of the shaft. In one embodiment, the jaws may remain approximately parallel to one another as the jaws are opened and closed.

In accordance with another aspect of the present invention, a method for ablating tissue is provided using an apparatus including a shaft and a clamp assembly carried by a distal end of the shaft. An articulating section of the shaft may be unlocked, articulated into a desired shape, and locked. With the articulating section locked in the desired shape, the clamp assembly may be inserted into a patient, tissue inside the patient may be clamped using the clamp assembly, and electrical energy may be delivered from the clamp assembly, e.g., to create a lesion.

In accordance with another aspect of the present invention, a method for ablating tissue may use a clamp assembly that includes a shaft carrying first and second jaws that remain substantially parallel to one another when the jaws are opened and/or closed. An articulating section of the shaft may be unlocked, articulated into a desired shape, and locked. With the articulating section locked in the desired shape, the clamp assembly may be inserted into a patient. The jaws may be opened, a tissue structure may be placed between the jaws, and the jaws may be closed to hold the tissue structure. The jaws remain substantially parallel to one another as the jaws are closed, thereby holding the tissue structure with a substantially uniform compressive force. Energy may then be delivered via one or more electrodes on the jaws to ablate or otherwise treat the tissue structure.

Other aspects and features of the invention may be evident from reading the following detailed description of the drawings, which is intended to illustrate, not limit, the invention.

### BRIEF DESCRIPTION OF THE DRAWINGS

Preferred embodiments of the present invention are illustrated by way of example, and not by way of limitation, in the figures of the accompanying drawings, in which like reference numerals refer to like components, and in which:

FIGS. 1A and 1B are side views of an apparatus including opposing jaws for clamping tissue therebetween;

FIG. 2 is a side view of an apparatus coupled to a source of energy, in accordance with an embodiment of the present invention;

FIGS. 3A and 3B are cross-sectional side views of a distal end of the apparatus of FIG. 2, including a clamp assembly in opened and closed positions, respectively;

FIG. 3C is a cross-sectional detail of the clamp assembly of FIG. 3A;

FIGS. 4A and 4B are cross-sectional side views of another embodiment of a clamp assembly, including opposing jaws in opened and closed positions, respectively;

FIG. 4C is a variation of the clamp assembly of FIGS. 4A and 4B, showing the jaws closed around a tissue structure;

FIGS. 5A and 5B are cross-sectional side views of yet another embodiment of a clamp assembly, including opposing jaws in open and closed positions, respectively;

FIGS. 6A and 6B are cross-sectional side views of still another embodiment of a clamp assembly, including opposing jaws in open and closed positions, respectively;

FIG. 7 is a perspective view of another variation of a clamp assembly;

FIGS. 8A and 8B are partial cross-sectional views of a chest, showing a method for placing a clamp assembly adjacent tissue to be treated; and

FIGS. 8C-8E are cross-sectional views of a tissue structure being clamped between jaws of a clamp assembly.

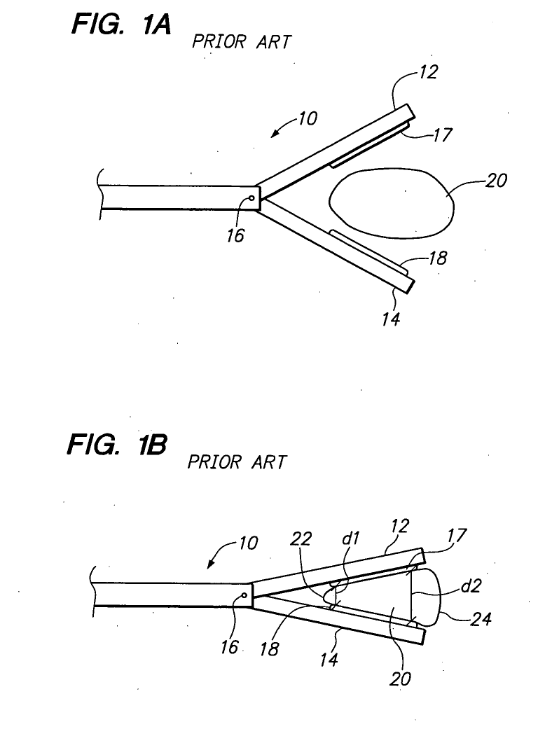

### DESCRIPTION OF THE PREFERRED EMBODIMENTS

Various embodiments of the present invention are described hereinafter with reference to the figures. It should be noted that the figures are not drawn to scale and that elements of similar structures or functions are represented by like reference numerals throughout the figures. It should also be noted that the figures are only intended to facilitate the description of specific embodiments of the invention. They are not intended as an exhaustive description of the invention or as a limitation on the scope of the invention. In addition, an illustrated embodiment need not have all aspects or advantages of the invention shown. An aspect or an advantage described in conjunction with a particular embodiment of the present invention is not necessarily limited to that embodiment and may be practiced in any other embodiments of the present invention even if not so illustrated.

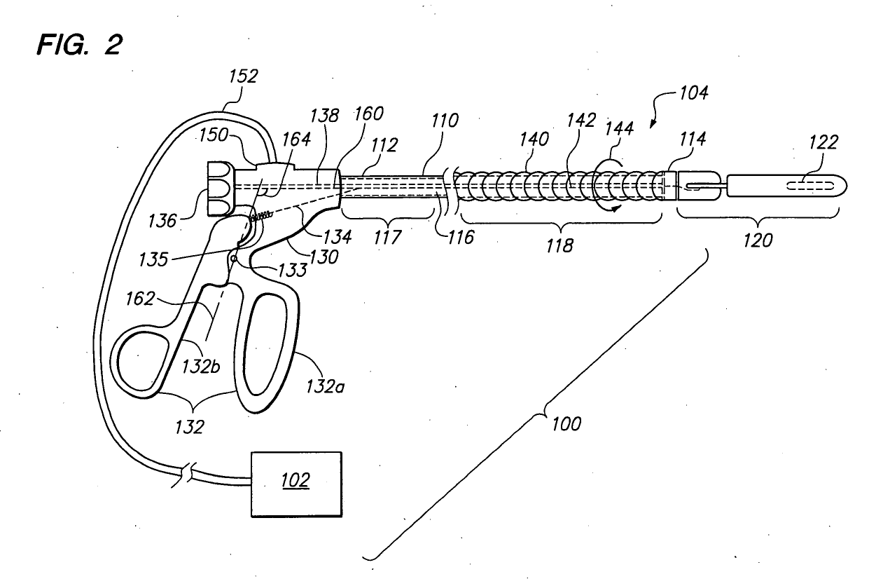

Referring to FIG. 2, a tissue ablation system 100 is shown that may include an energy source 102 and an ablation device 104. The energy source 102 is preferably a radio frequency (RF) generator, such as the EPT-1000 XP generator available from EP Technologies, Inc., San Jose, Calif. The ablation device 104 may include an elongate shaft 110 having a proximal end 112, a distal end 114, and a lumen 116 extending between the proximal and distal ends 112, 114. The ablation device 104 may also include a clamp assembly 120 carried by the distal end 114 of the shaft 110, one or more electrodes 122 carried by the clamp assembly 120, and a handle assembly 130 on the proximal end 112 of the shaft 110. The handle assembly 130 may have a connector 150 for coupling the ablation device 104 to the energy source 102, e.g., via a cable 152, which may provide electrical energy to the ablation device 104. Alternatively, the cable 152 may be secured to the ablation device 104, in which case, the ablation device 104 may not include the connector 150.

The shaft 110 may include one or more non-bendable sections 117 that may be substantially rigid and/or malleable, and one or more bendable or articulating sections 118 that may allow the shaft to be customized into a desired shape or profile during a procedure. The non-bendable section(s) 117 of the shaft 110 may be made from one or more materials, e.g., a metal, such as stainless steel, a polymer or other plastic, such as PEEK or polycarbonate, and/or a composite material.

For example, as shown in FIG. 2, the shaft 110 may include an articulating section 118 having a length between about half and forty centimeters (0.5-40 cm), and preferably, between about one and ten centimeters (1-10 cm), and a cross-sectional dimension between about two and 22 French (0.67-7.33 mm). However, the articulating section 118 may also have other lengths and/or cross-sectional sizes, depending upon the particular application. The articulating section 118 may be made from a plurality of segments 140 connected to one another, e.g., via ball-bearing connections and/or an internal filament 138. The filament 138 can be a wire, a cable, or a suitable elongate member.

Such construction may allow the articulating section 118 to be articulated in various directions such that a desired shape or configuration of the shaft 110 may be obtained. Such construction may also allow one of the segments 140 to be rotated relative to another of the segments 140, thereby enabling a portion of the articulating section 118 to be rotated (or twisted) relative to a remaining portion of the articulating section 118, e.g., about a longitudinal axis 142 (as indicated by arrow 144 in FIG. 2).

Alternatively, the articulating section 118 may be made from polymer rings, similar to those used for air or water nozzles, or from tube sections that are commonly used for fiber optic light wands. In a further alternative, a bendable section may be provided that is made from a malleable metal, such as aluminum, that is sufficiently flexible to be bent, but sufficiently stiff or rigid to retain a configuration into which it is bent. The bendable or articulating section 118 may also have other types of construction that may be shaped into a desired form or configuration.

The handle assembly 130 includes a knob 136 for applying tension to the filament 138. The filament 138 is disposed within the lumen 116 of the shaft 110, and is connected between the knob 136 and the distal end 114 of the shaft 110. The distal end of the filament 138 can be secured to the distal end 114 of the shaft 110 by a weld, a suitable adhesive, a screw, a mechanical anchor, or other types of securing mechanisms. During use, the knob 136 can be turned in one direction to reduce a tension of the filament 138, thereby allowing the bendable section 118 to be easily bent. After a desired shape or profile of the bendable section 118 has been created, the knob 136 can be turned in the opposite direction to increase the tension of the filament 138. The tensioning of the filament 138 causes the shaft 110 to undergo compression, thereby stiffening or locking the shaft 110 in its bent configuration.

The handle assembly 130 may also include an actuator for operating the clamp assembly 120. For example, in one embodiment, the actuator may include an actuating device 132, and a control wire 134 secured between the actuating device 132 and the clamp assembly 120 (FIG. 2). The actuating device 132 may be used for applying tension to a control wire 134 to thereby control an operation of the clamp assembly 120. In alternative embodiments, other structures, such as a rod or a spring may be used instead of the control wire 134. The actuating device 132 has an axis 162 that forms an angle 164 with a longitudinal axis 160 of the shaft 110. In the illustrated embodiment, the angle 164 is approximately 100°. However, in alternative embodiments, the angle 164 can be anywhere between 45° and 180°. The control wire 134 is disposed within the lumen 116 of the shaft 110, and is secured between the actuating device 132 and the clamp 120. In the illustrated embodiment, the actuating device 132 has a first portion 132a and a second portion 132b that is movable relative to the first portion 132a about a connecting pin 133. A proximal end of the control wire 134 is secured to a spring 135, which in turn, is anchored or secured to the second portion 132b of the actuating device 132. The spring 135 reduces the amount of tension that can be applied to the control wire 134, and therefore, a clamping force that can be applied by the actuating device 132 to the clamp assembly 120. In an alternative embodiment, the spring 135 is optional and the ablation device 104 does not include the spring 135.

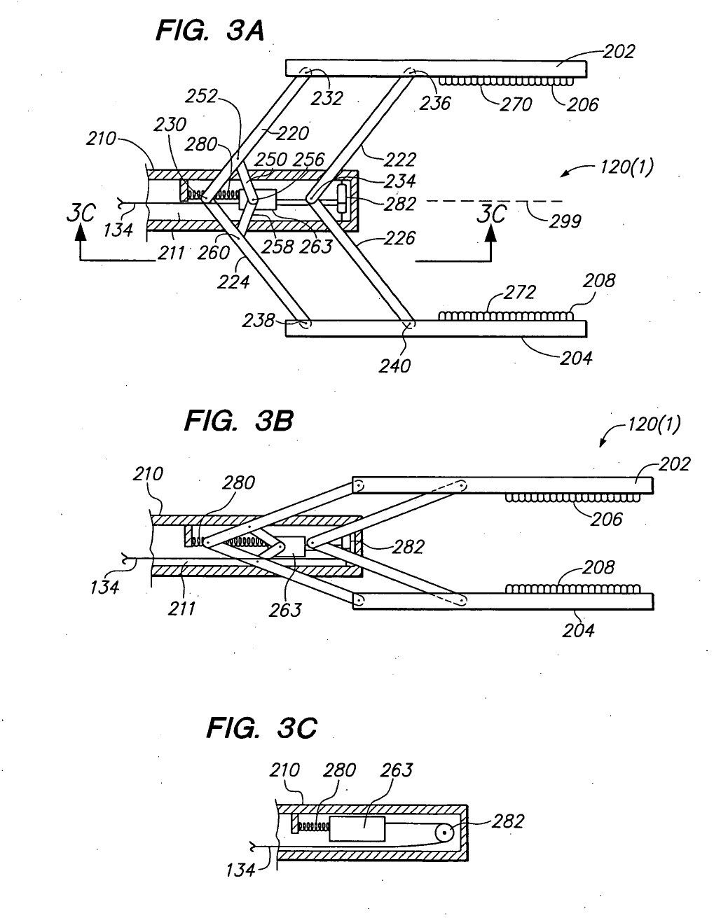

The clamp assembly 120 may be constructed in a variety of ways. For example, FIGS. 3A-3C illustrate one embodiment of a clamp assembly 120(1) that includes an elongate tubular member 210 and first and second opposing jaws 202, 204 carried on a distal end of the tubular member 210. The tubular member 210 may extend from or be part of a shaft (not shown), such as the shaft 110 shown in FIG. 2. Returning to FIGS. 3A and 3B, the first and second jaws 202, 204 may include first and second electrodes 206, 208, respectively, that may be arranged opposite one another for delivering energy to tissue held between the jaws 202, 204. The tubular member 210 may include a lumen 211, through which the control wire 134 extends, e.g., from a handle assembly, such as the handle assembly 130 shown in FIG. 2.

With continued reference to FIGS. 3A and 3B, the control wire 134 may be coupled to a linkage including a plurality of supports or other linkage members 220, 222, 224, 226, 250, 258 for moving one or both of the jaws 202, 204 relative to the tubular member 210 and/or one another. In the illustrated embodiment, the linkage members 220 and 222 connect the first jaw 202 to the tubular member 210, and the linkage members 224 and 226 connect the second jaw 204 to the tubular member 210. The linkage member 220 is rotatably secured at one end to the tubular member 210 by a pin 230, and at the other end to the first jaw 202 by a pin 232. The linkage member 222 is rotatably secured at one end to the tubular member 210 by a pin 234, and at the other end to the first jaw 202 by a pin 236. The linkage member 224 is rotatably secured at one end to the tubular member 210 by the pin 230, and at the other end to the second jaw 204 by a pin 238. The linkage member 226 is rotatably secured at one end to the tubular member 210 by the pin 234, and at the other end to the second jaw 204 by a pin 240. The linkage members 250 and 258 connect the linkage members 220 and 224, respectively, to an actuating member 263, and function to rotate the linkage members 220 and 224 in response to movement of the actuating member 263. The linkage member 250 is rotatably secured at one end to the linkage member 220 by a pin 252, and at the other end to the actuating member 263 by a pin 256. The linkage member 258 is rotatably secured at one end to the linkage member 224 by a pin 260, and at the other end to the actuating member 263 by the pin 256. Thus, when the control wire 134 is subjected to proximal tension or otherwise moved proximally, the linkage elements 250, 258 may cause the jaws 202, 204 to move from an open position, shown in FIG. 3A, towards one another in a closed position, shown in FIG. 3B. The supports and linkage elements 220, 222, 224, 226, 250, 258 may be made from a variety of materials, such as metals or metal alloys, plastics, and/or polymers.

The linkage may be biased to move the jaws 202, 204 towards the opened. configuration, e.g., by coupling the distal end of the control wire 134 to one end of a spring 280 that has its other end fixed relative to the tubular member 210. Optionally, as best seen in FIG. 3C, the control wire 134 may loop around a roller 282 before being coupled to the spring 280, as shown, e.g., to reduce the force necessary to move the jaws 202, 204. Alternatively, the spring 280 may be connected directly to the distal end of the tubular member 210, and the roller 282 may be eliminated;

The first and second jaws 202, 204 and/or the electrodes 206, 208 may carry one or more temperature sensor(s), thermocouples, thermisters, and the like (not shown), for sensing a temperature of respective electrodes 266, 208 and/or tissue contacted by the electrodes 206, 208. Each of the first and second electrodes 206, 208 may have a length between half and ten centimeters (0.5-10.0 cm), and preferably between about two and seven centimeters (2.0-7.0 cm), and a width between about one and five millimeters (1-5 mm), and preferably about one and three millimeters (1-3 mm). Alternatively, the first and the second electrodes 206, 208 may have other shapes and dimensions other than the linear configuration shown, e.g., curved in one or more planes that extend parallel to the longitudinal axis 299.

In the illustrated embodiment, each of the first and second electrodes 206, 208 may be an elongated coil that extends along the respective jaws 202, 204. However, the first and second electrodes 206, 208 may have other shapes and/or constructions capable of delivering energy to tissue in a desired manner. The electrodes 206, 208 may be made from a material that has both a relatively high electrical conductivity and a relatively high thermal conductivity, such as gold and platinum. Noble metals are preferred. One or more leads (not shown) may extend from the electrodes 206, 208, through the lumen 211 of the tubular member 210 and the lumen 116 of the shaft 110 (not shown, see FIG. 2) to electrically couple the electrodes 206, 208 to the electrical connector 150 on the handle assembly 130 (or to the cable 152).

The first and second electrodes 206, 208 may be operated in a bipolar mode to deliver energy to tissue between the electrodes 206, 208, e.g., to ablate tissue. Alternatively, the electrodes 206, 208 may be operated in a monopolar mode, with the electrodes 206, 208 connected to an active terminal of a generator, and a passive and/or dispersive electrode (not shown) connected to a return terminal of the generator and fixed to a body location remote from the tissue site being treated.

The first and second jaws 202, 204 may have a substantially linear profile such that they may be inserted through a trocar or a cannula (not shown) before or during use. Alternatively, the first and second jaws 202 and 204 may have a curvilinear profile, a slight bent configuration, or other shape. The first and second jaws 202, 204 may be made from a variety of materials, such as metals or metal alloys, plastics, and/or polymers. If the first and the second jaws 202, 204 are made from an electrical conductive material, the first and the second electrodes 206, 208 are preferably electrically isolated from the first and the second jaws 202, 204, respectively. In the illustrative embodiment, the first and the second jaws 202, 204 may be made from a material that is relatively rigid. Alternatively, at least a portion of the first jaw 202 and/or the second jaw 204 may be made from a malleable material allowing a physician to bend the first and/or second jaws 202, 204 into desired shape(s) during use.

When tension is applied to the control wire 134, the actuating member 263 is pulled distally, separating a distance between the pins 230 and 256, which in turn causes the first and second jaws 202 and 204 to move towards each other. When the tension in the control wire 134 is reduced or removed, the spring 280 pulls the actuating member 263 proximally, bringing the pins 230 and 256 closer to each other, which in turn causes the first and second jaws 202 and 204 to move further apart from each other. Such configuration is advantageous in that the first jaw 202 can move approximately in parallel relative to the second jaw 204, thereby allowing the clamp assembly 120(1) to evenly hold or compress a tissue of any size.

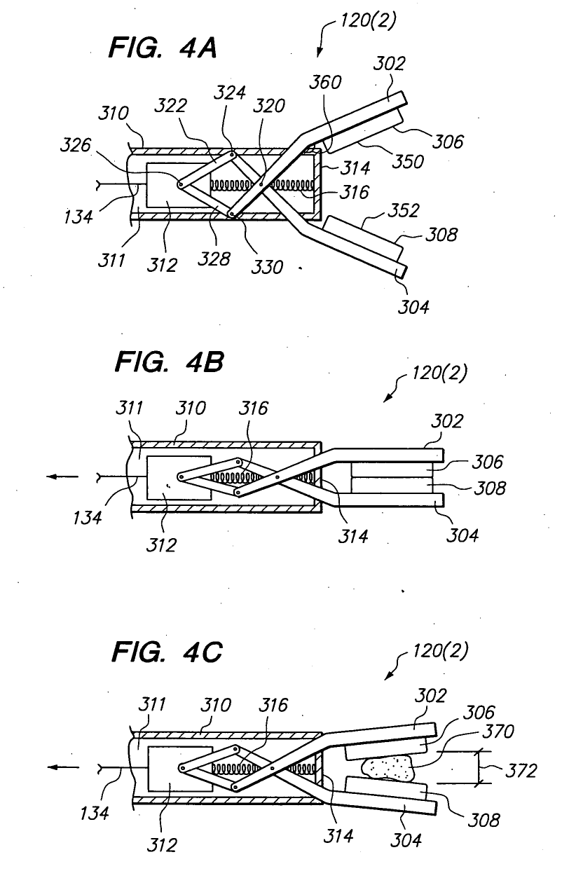

FIGS. 4A and 4B illustrate another embodiment of a clamp assembly 120(2) that includes a first jaw 302 carrying a first electrode 306, a second jaw 304 carrying a second electrode 308, a body 310 having a lumen 311, an actuating member 312 disposed within the lumen 311 of the body 310, and linkage members 322 and 328. The first and second jaws 302 and 304 are rotatably secured to the body 310 by a pin 320. The linkage member 322 is rotatably secured at one end to the second jaw 304 by a pin 324, and at the other end to the actuating member 312 by a pin 326. The linkage member 328 is rotatably secured at one end to the first jaw 302 by a pin 330, and at the other end to the actuating member 312 by the pin 326. The actuating member 312 is slidable within the lumen 311 of the body 312, and is secured at one end to the control wire 134, and at the other end to a distal end 314 of the body 310 via a spring 316.

During use, the clamp assembly 210(2) may be opened by releasing the tension at the control wire 134, thereby allowing the spring 316 to pull the actuating member 312 distally towards the distal end 314 of the body 310. Distal movement of the actuating member 312 causes the pin 326 to move closer to the pin 320, which in turn, causes the clamp assembly 210(2) to open. When tension is applied to the control wire 134, the wire 134 pulls the actuating member 312 proximally, thereby causing the pins 326 and 320 to move further apart from, each other, which in turn, causes the clamp assembly 210(2) to close (FIG. 4B). As shown in FIG. 4B, when the clamp assembly 210(2) is closed, a contacting surface 350 of the first electrode 306 is approximately parallel to a contacting surface 352 of the second electrode 308. This can be accomplished by choosing a desired shape of the first and second jaws 302 and 304 (e.g., by selecting an appropriate angle 360), and/or by varying a dimension of the linkage members 322 and 328. Alternatively, as shown in FIG. 4C, the clamp assembly 210(2) can have a configuration such that when it holds on to a tissue 370 having a compressed thickness 372, the contacting surface 350 of the first electrode 306 is approximately parallel to the contacting surface 352 of the second electrode 308.

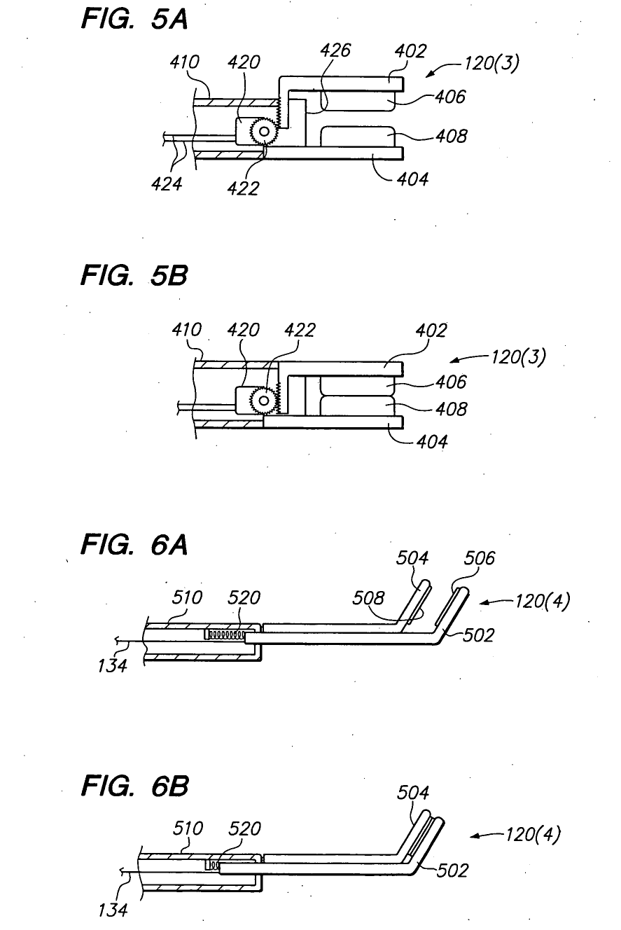

FIGS. 5A and 5B illustrate another embodiment of a clamp assembly 120(3). The clamp assembly 120(3) includes a first jaw 402 carrying a first electrode 406 and having an extension 426, a second jaw 404 carrying a second electrode 408, a body 410, a motor 420 carried by the body 410, and electrical wires 424 for supplying energy to the motor 420. The motor 420 includes a gear 422 that is engaged with the saw-teeth of the extension 426. A rotation of the gear 422 in one direction causes the first jaw 402 to move closer to the second jaw 404, thereby closing the clamp assembly, 120(3), and a rotation of the gear 422 in an opposite direction causes the first jaw 402 to move further from the second jaw 404, thereby opening the clamp assembly 120(3). Such configuration is advantageous in that the first jaw 402 can move approximately in parallel relative to the second jaw 404, thereby allowing the clamp assembly 120(3) to evenly hold or compress a tissue of any size. In this case, the ablation device 104 does not include the control wire 134, and the handle assembly 130 does not include the actuating device 132. Instead, the handle assembly 130 includes a switch (not shown) operable in a first position and a second position. Placement of the switch in the first position causes the motor 420 to rotate in one direction, and placement of the switch in the second position causes the motor 420 to rotate in an opposite direction.

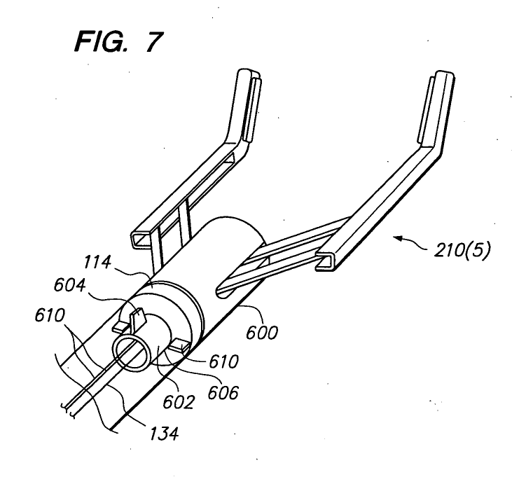

FIGS. 6A and 6B illustrate another embodiment of a clamp assembly 120(4). The clamp assembly 120(4) includes a first jaw 502 carrying a first electrode 506, a second jaw 504 carrying a second electrode 508, and a body 510. The second jaw 504 is secured to the body 510 and the first jaw 502 is slidable relative relative to the body 510. The control wire 134 is secured to the first jaw 502, and can be used to control a position of the first jaw 502. The clamp assembly 120(4) also includes a spring 520 secured to the first jaw 502. Proximally pulling the control wire 134 positions the first jaw 502 closer to the second jaw 504, thereby closing the clamp assembly 120(4). When a tension in the control wire 134 is reduced or removed, the spring 520 pushes the first jaw 502 distally to open the clamp assembly 120(4). Such configuration is advantageous in that the first jaw 502 can move approximately in parallel relative to the second jaw 504, thereby allowing the clamp assembly 120(3) to evenly hold or compress a tissue of any size.

It should be noted that the clamp assembly 120 should not be limited to the examples discussed previously, and that the clamp assembly 120 may have other configurations including a closed configuration for holding tissue, and an opened configuration for releasing the tissue.

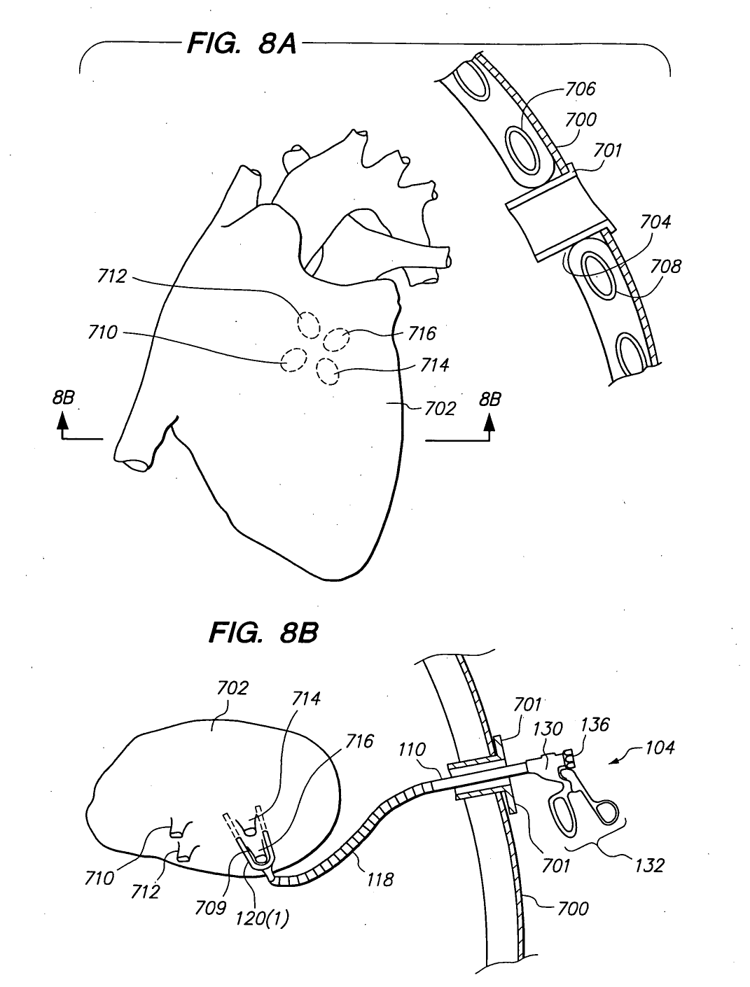

In the previously described embodiments, the clamp assembly 120 is fixed to the distal end 114 of the shaft 110. However, this need not be the case. For example, FIG. 7 shows a variation of a clamp assembly 120(5) that is rotatably secured to the distal end 114 of the shaft 110. The clamp assembly 120(5) includes a body 600 having an tubular portion 602 that is sized to mate with an opening 606 at the distal end 114 of the shaft 110. The tubular portion 602 is rotatable relative to the distal end 114 of the shaft 110. The distal end 114 of the shaft 110 can optionally include one or more stoppers 610 for engaging with a protrusion 604 on the tubular portion 602, thereby limiting a range of rotation that the clamp assembly 120(5) can have relative to the distal end 114 of the shaft 110. The control wire 134 and the ablation wires 610 run through the lumen of the tubular portion 602 and extend through the lumen 116 of the shaft 110.

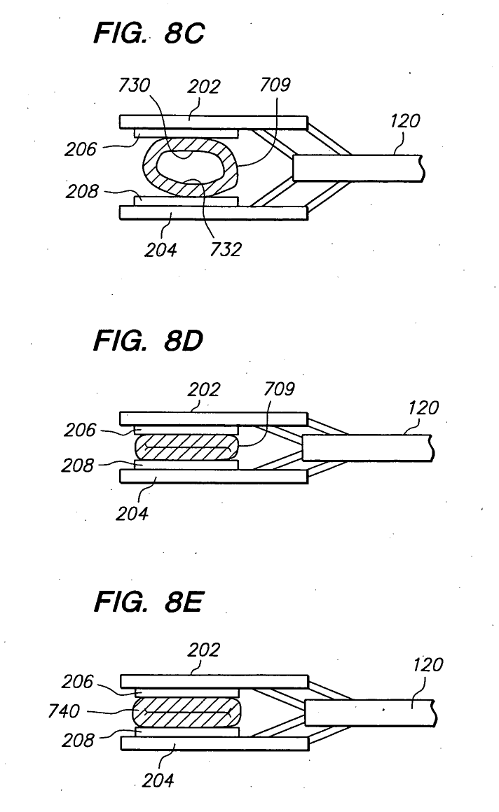

Referring to FIGS. 8A-8D, a method is shown for using an apparatus, such as those described above, for treating tissue, e.g., for cardiac ablation therapy. Although the method is described generally with reference to the embodiment of the ablation device 104 having the clamp assembly 120(1) shown in FIG. 2, a person skilled in the art will appreciate that the methods described may also apply to other embodiments of the ablation device 104 and/or clamp assembly 120 previously described, or even embodiments not described herein.

Initially, an incision may be created into a patient's skin 700 to form an opening 704. For example, a small incision or port in the intercostals space or subxiphoid may be created by a trocar 701 to access the patient's heart 702 (FIG. 8A). In the illustrated embodiment, the trocar 701 may be placed between a first rib bone 706 and a second rib bone 708. Other procedures, such as a minimally invasive direct coronary artery bypass (MIDCAB) procedure, or a conventional thoracotomy or a thorascopic technique, may also be used to access a target tissue at or proximate the heart 702.

Before the clamp assembly 120(1) is inserted into the patient, the articulating or bendable section(s) 118 of the shaft 110 may be articulated or bent until a desired configuration of the shaft 110 is achieved. If the clamp assembly 120(1) is rotatable relative to the shaft 110 (such as that shown in FIG. 7), the clamp assembly 120(1) may also be rotated relative to the shaft 110 to obtain a desired configuration of the ablation device 104. A desired configuration of the shaft 110 may be determined based on the relative location of the entry point (i.e., the opening 704) and the location of a target tissue. For the purpose of the following discussion, it will be assumed that a lesion is to be formed at an atrial tissue site 709, e.g., at the ostium of the left inferior pulmonary vein (LIPV) 716. Based upon the relative locations of the target tissue and the opening 704, the shape and/or configuration of the articulating or bendable section 118 may be changed such that, when the ablation device 104 is inserted through the opening 704, the clamp assembly 120(1) may reach the target tissue site. As such, by having a articulating or bendable shaft 110, a profile of the shaft 110 may be created such that the clamp assembly 120(1) may reach the target tissue site regardless of where the opening 704 is. After a desired shape of the shaft 110 has been obtained, the knob 136 or other actuator may be operated to create or increase a tension in the filament 138 to stiffen and/or lock the shaft 110 into the desired shape.

The distal end of the ablation device 104 may be inserted through the trocar 701, and advanced until the clamp assembly 120(1) is adjacent the atrial tissue site 709 of the heart 702 (FIG. 8B). The ablation catheter 104 may be manipulated to place the clamp assembly 120(1) in close proximity to the atrial tissue site 709 that is targeted for ablation. For example, the handle assembly 130 may be twisted or moved axially to position the clamp assembly 120(1). In the illustrated embodiment, the clamp assembly 120(1) is positioned at the posterior of the heart,802 for ablation of the atrial tissue site 709.

The clamp assembly 120(1) may then be opened, e.g., by operating the actuating device 132. Particularly, the second portion 132b of the actuating device 132 may be moved apart from the first portion 132a of the actuating device 132 to release a tension in the control wire 134, thereby allowing the spring 280 to pull the first and second jaws 202 and 204 into an opened configuration. The clamp assembly 120(1) is then positioned such that the atria tissue 709 is between the first jaw 202 and the second jaw 204 of the clamp assembly 120(1). The second jaw 204 is then caused to move relative to the first jaw 202 (i.e., by operating on the actuating device 132) to close the clamp assembly 120, thereby holding the atria tissue 709 between the first and second electrodes 206 and 208 (FIG. 8C). Although only the LIPV 716 is shown to be clamped by the clamp assembly 120, in an alternative embodiment, if the jaws of the clamp assembly 120(1) is long enough, both the RIPV 714 and LIPV 716 can be clamped simultaneously by the clamp assembly 120(1) (see dashed lines representing an extension of the jaws of the clamp assembly 120(1) in FIG. 8B). If desired, the atria tissue 709 can be further compressed by applying additional tension force to the control wire 134. In one embodiment, the atria tissue 709 can be compressed by the clamp assembly 120(1) such that a first wall 730 on one side of the ostium of the LIPV 716 is in contact with a second wall 732 on an opposite side of the ostium of the LIPV 716 (FIG. 8D). In the illustrated embodiment, the second jaw 204 remains approximately parallel to the first jaw 202 as the second jaw 204 is moved relative to the first jaw 202. This has the benefit of ensuring that the atria tissue 709 is uniformly compressed across its section, which allows ablation energy to be uniformly delivered across the section of the atria tissue 709.

Next, with the ablation device 104 coupled to the RF generator 102, ablation energy is delivered from the generator 102 to the electrodes 206 and 208 of the ablation catheter 104. By clamping the target tissue and compressing the target tissue between the first and second electrodes 206 and 208, the path of the current delivered by the first electrode 206 to the second electrode 208 is shorter, i.e., RF energy is directed from the first electrode 206, across the target tissue, and to the electrode 208, thereby efficiently forming a transmural lesion 740 at the target tissue (FIG. 8E). Particularly, the transmural lesion 740 is formed at the first wall 730 and at the second wall 732 of the atria tissue 709. Such configuration also allows the target tissue to be ablated without a significant dissipation of RF energy to adjacent tissues.

After the lesion 740 at the atria tissue has been created, the clamp assembly 120(1) is opened to release the atria tissue 709 by manipulating the actuating device 132. If desired, the above described procedure can be repeated to create additional lesions at other atria tissue (i.e., at the ostium of the right superior pulmonary vein (RSPV) 710, at the ostium of the left superior pulmonary vein (LSPV) 712, and at the ostium of the right inferior pulmonary vein (RIPV) 714). If the jaws of the clamp assembly 120(1) is long enough, the clamp assembly 120(1) can be used to clamp the RSPV 710 and the LSPV 712 and create additional lesions at these sites simultaneously. The physician can bend the shaft 110 into different shapes such that the clamp assembly 120(1) can reach different target tissues when inserted into the patient. This is advantageous in that it obviates the need to use multiple ablation devices having different predetermined configurations for reaching different target tissues. When all desired lesions have been created, the clamp assembly 120(1) is then retracted and removed from the interior of the patient.

Although several embodiments of the ablation device 104 have been described, it should be noted that the scope of the invention should not be so limited, and that variations and modifications of the previously described embodiments are intended to be within the scope of the invention. For example, instead of carrying electrodes that operate in a bipolar arrangement, in alternative embodiments, any of the clamp assemblies 120 described previously (or clamp assemblies that have not been described) may carry one or more electrodes that operate in a monopolar arrangement. In this case, the system 100 may include an indifferent patch electrode or ground pad that may be coupled to the energy source 102. An ablation procedure using a monopolar arrangement may include placing the indifferent patch electrode on the patient's skin. Electrical energy is directed from the electrodes carried by the clamp assembly 120 through the patient's body to the indifferent patch electrode that is electrically coupled to a ground or return terminal on the energy source 102, thereby completing the energy path. During use, electrical energy may flow from the electrodes on the clamp assembly to the patch electrode.

Also, instead of the handle assembly 130 shown previously, in an alternative embodiment, the ablation device 104 may include a handle assembly of different constructions capable of performing the functions described herein. For example, instead of the knob 136 shown in FIG. 2, the handle assembly 130 may include other devices or mechanisms for adjusting a tension in the filament 138. Also, instead of the actuating device 132 described previously, in alternative embodiments, the handle assembly 130 may include an actuating device having a shape and/or configuration different from those shown previously. For example, the handle assembly 130 may include a button, a knob, a trigger, or other types of device for allowing a user to control an operation of the clamp assembly 120.

Furthermore, although the above described system and method have been described in the context of creating a lesion at the atrial tissue, it should be understood that the ablation device 104 may also be used in many different environments and/or applications. For example, the ablation device 104 may also be used to create lesions, such as transmural lesions, at other locations of the heart or at different locations within the body.

Thus, although different embodiments have been shown and described, it will be apparent to those skilled in the art that many changes and modifications may be made thereunto without the departing from the scope of the invention, which is defined by the following claims and their equivalents.

1. An ablation device, comprising:
a shaft comprising proximal and distal ends, at least a portion of the shaft being bendable to form a desired configuration;
a clamp assembly on the distal end of the shaft, the clamp assembly comprising first and second opposing jaws, at least one of the first and second jaws being moveable relative to the other to open and close the clamp assembly;
first and second electrodes on the first and second jaws, respectively; and
a handle on the proximal end of the shaft.

2. The ablation device of claim 1, wherein at least a portion of the shaft is capable of being rotated about a longitudinal axis of the shaft.

3. The ablation device of claim 1, wherein the shaft comprises a first segment and a second segment rotatably secured to the first segment.

4. The ablation device of claim 3, wherein the second segment is rotatably secured to the first segment by a ball-bearing connection.

5. The ablation device of claim 1, wherein the shaft comprises one or more polymer rings.

6. The ablation device of claim 1, wherein the at least a portion of the shaft is made from a malleable material.

7. The ablation device of claim 1, wherein the shaft further comprising a lumen extending between the proximal and distal ends.

8. The ablation device of claim 7, further comprising:
a tensioning device located proximate to the proximal end of the shaft; and
a wire having a proximal end secured to the tensioning device and a distal end secured to the distal end of the shaft, at least a portion of the wire is disposed within the lumen of the shaft;
wherein the tensioning device is operable to create or adjust a tension in the wire.

9. The ablation device of claim 8, wherein the tensioning device comprises a knob.

10. The ablation device of claim 7, further comprising:
an actuating device coupled to the handle; and
a control wire having a proximal end coupled to the actuating device and a distal end secured to the clamp assembly, at least a portion of the control wire is disposed within the lumen of the shaft;
wherein the actuating device is operable to create or adjust a tension in the control wire.

11. The ablation device of claim 10, wherein the actuating device comprises a first portion and a second portion rotatably secured to the first portion.

12. The ablation device of claim 10, further comprising a spring secured between the proximal end of the control wire and the actuating device.

13. The ablation device of claim 1, wherein the clamp assembly is rotatably secured to the distal end of the shaft.

14. The ablation device of claim 1, wherein the first electrode has a first surface, the second electrode has a second surface, and the clamp assembly having a configuration such that when a tissue is compressed between the first and second jaws, the first surface of the first electrode is approximately parallel to the second surface of the second electrode.

15. The ablation device of claim 1, wherein the second jaw remains approximately parallel to the first jaw as the second jaw is moved relative to the first jaw.

16. The ablation device of claim 1, wherein at least a portion of the first jaw is capable of being bent into a desired shape.

17. The ablation device of claim 1, wherein the first and the second electrodes operate in a bipolar arrangement.

18. The ablation device of claim 1, wherein the first and the second electrodes operate in a unipolar arrangement.

19. An ablation device, comprising:
a shaft having a proximal end and a distal end;
a clamp assembly secured to the distal end of the shaft, the clamp assembly having a first jaw and a second jaw, the second jaw moveable relative to the first jaw to open or close the clamp assembly, the second jaw remains approximately parallel to the first jaw as the second jaw is moved relative to the first jaw;
a first electrode secured to the first jaw of the clamp assembly;
a second electrode secured to the second jaw of the clamp assembly; and
a handle connected to the proximal end of the shaft.

20. The ablation device of claim 19, wherein the shaft further having a lumen extending between the proximal end and the distal end.

21. The ablation device of claim 20, further comprising:
an actuating device coupled to the handle; and
a control wire having a proximal end coupled to the actuating device and a distal end secured to the clamp assembly, at least a portion of the control wire is disposed within the lumen of the shaft;
wherein the actuating device is operable to create or adjust a tension in the control wire.

22. The ablation device of claim 21, wherein the actuating device comprises a first portion and a second portion rotatably secured to the first portion.

23. The ablation device of claim 21, further comprising a spring secured between the proximal end of the control wire and the actuating device.

24. The ablation device of claim 19, wherein the clamp assembly is rotatably secured to the distal end of the shaft.

25. The ablation device of claim 19, wherein at least a portion of the shaft is capable of being rotated about a longitudinal axis relative to a remaining portion of the shaft.

26. The ablation device of claim 19, wherein at least a portion of the first jaw is capable of being bent into a desired shape.

27. The ablation device of claim 19, wherein the first and the second electrodes operate in a bipolar arrangement.

28. The ablation device of claim 19, wherein the first and the second electrodes operate in a unipolar arrangement.

29. A method of ablating a tissue, comprising:
providing an ablation device having a shaft and a clamp assembly connected to the shaft, the clamp assembly carrying at least one electrode;
bending the shaft of the ablation device into a desired shape;
inserting at least a portion of the ablation device into a patient;
clamping a tissue inside the patient using the clamp assembly; and
delivering ablation energy to the tissue via the at least one electrode.

30. The method of claim 29, further comprising locking the shaft in the desired shape.

31. The method of claim 30, wherein the locking comprises creating a compression in the shaft.

32. The method of claim 29, wherein the inserting the at least a portion of the ablation device into the patient comprises placing the clamp assembly through a trocar or a cannula.

33. The method of claim 29, wherein the clamp assembly having a first jaw and a second jaw, and the clamping comprises causing the second jaw to move relative to the first jaw, wherein the second jaw remains approximately parallel to the first jaw as the second jaw is moved relative to the first jaw.

34. A method of ablating a tissue, comprising:
providing a clamp assembly, the clamp assembly having a first jaw carrying a first electrode, and a second jaw carrying a second electrode, the first electrode having a first surface, and the second electrode having a second surface;
holding a tissue inside a patient using the clamp assembly such that the first surface of the first electrode is approximately parallel to the second surface of the second electrode when the tissue is secured between the first and second electrodes; and
delivering ablation energy to the first and second electrodes.

35. The method of claim 34, wherein the tissue comprises at least a portion of an atria tissue, and the method further comprising compressing the at least a portion of the atria tissue.

36. The method of claim 35, wherein the compressing is performed such that a first wall of the at least a portion of the atria tissue is in contact with a second wall of the at least a portion of the atria tissue.

37. The method of claim 36, wherein the delivering the ablation energy is continued until a transmural lesion is created at the first wall and at the second wall.

38. The method of claim 34, wherein the clamping comprises moving the second jaw relative to the first jaw, the second jaw remains approximately parallel to the first jaw as the second jaw is moved relative to the first jaw.

39. The method of claim 34, wherein the delivering the ablation energy is continued until a transmural lesion is created in the tissue.
